(19) United States
(12) Patent Application Publication
(10) Pub. No.: US 2022/0370613 A1
(43) Pub. Date: Nov. 24, 2022

(54) PHARMACEUTICAL COMPOSITION

(71) Applicant: Renown Pharma Inc., Mill Valley, CA (US)

(72) Inventors: Anthony CLARKE, Checkendon, Reading, Berkshire (GB); Jagdeep SHUR, Somerset, Bath and North East Somerset (GB)

(21) Appl. No.: 17/640,625

(22) PCT Filed: Sep. 4, 2020
(86) PCT No.: PCT/IB2020/058246

§ 371 Date: Mar. 4, 2022

Related U.S. Application Data
(60) Provisional application No. 62/895,619, filed on Sep. 4, 2019.

(30) Foreign Application Priority Data
Sep. 4, 2019 (GB) 1912686.1

(51) Int. Cl. A61K 47/06; A61K 47/10; A61K 9/14

(52) U.S. Cl. CPC A61K 47/06; A61K 47/10; A61K 9/14

(57) ABSTRACT

Compositions and devices comprising apomorphine or pharmaceutically acceptable salts are described. The compositions may comprise a solution of apomorphine, or a pharmaceutically acceptable salt thereof, and a propellant. The solution may be a non-aqueous solution or an aqueous solution comprising degassed water. Devices may be configured to deliver compositions in form of particles or droplets having a mass median aerodynamic diameter (MMAD) or volume median diameter (VMD) greater than 10 pm; and/or a fine particle fraction (FRF) less than 30%.

This application claims priority to U.S. Provisional Application No. 62/895,619 filed on 4 Sep. 2019, the entire contents of each of which is incorporated herein by reference and relied upon.

The present invention relates to pharmaceutical compositions comprising apomorphine and uses thereof, such as compositions for use in treating Parkinson's disease or Male Erectile Dysfunction by buccal or sublingual administration.

### BACKGROUND OF THE INVENTION

Parkinson's disease is a chronic, progressive neurological disorder affecting approximately 20 in every 100,000 people. The disease is typically characterised by resting tremor, muscle rigidity, bradykinesia and postural instability. Although the exact pathological course of Parkinson's disease is unknown, the dopaminergic neurones in the substantia nigra are progressively destroyed which leads to a net decrease in the amount of dopamine in the basal ganglia. Dopamine replacement with levodopa is the current primary therapy for Parkinson's disease.

After a three to five year period of control, 25% of Parkinson's disease sufferers can develop “on-off” fluctuations. These are characterised by periods of a few minutes to a few hours during which the patient is able to move and walk easily (“on”), alternating with periods during which the patient experiences severe akinesia (“off”). Many patients also experience other unpleasant “off” period phenomena, such as depression, anxiety, panic, pain, delusions and dystonia, which follow a time-course parallel to the motor stage. The “off” periods may appear several times a day even when anti-parkinsonian drugs are given at the optimum dosage.

Dopamine agonists have been shown to decrease dyskinesias and “on-off” fluctuations when combined with levodopa therapy. Apomorphine is a non-ergot dopamine agonist which has a high affinity for D₂, D₃ and D₄ and lower affinity for D₁, and D₅ receptors. It has the following structural formula:

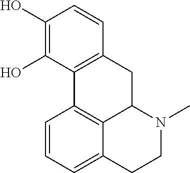 [SMILES: CN1CCc2cccc3c2C1Cc1ccc(O)c(O)c1-3]

When administered orally and swallowed, apomorphine is rapidly and extensively metabolised on “first-pass” through the liver, and very little unmetabolized drug reaches the circulation. The administration of high oral doses of apomorphine have been given in order to attempt to overcome this metabolism. Oral doses in excess of 500 mg apomorphine have been shown to produce a dose-dependent improvement in tremor, rigidity, and akinesia but are associated with drug-induced nephrotoxicity. This is thought to be a result of nephrotoxic metabolites produced by the liver, presumably due to extensive first-pass metabolism.

Subcutaneous injections of apomorphine have proven to be effective in the treatment of “on-off” fluctuations in Parkinson's disease within 5 to 15 minutes, and last for 45 to 90 minutes. Trials have shown consistent reversal of “off” period akinesia, a decrease in daily levodopa requirements and consequently a decrease in the amount of “on” period dyskinesias. Advantages over other dopamine agonists include a quick onset of action and lower incidence of psychological complications. For a “rescue therapy” in patients with “on-off” fluctuations, apomorphine also has the advantage over other dopamine agonists that it has a relatively short half-life.

As there is a large inter-subject variation in pharmacokinetics, patients undergo an initial dose titration period at the start of treatment. Nausea and vomiting which may occur as a result of the action of apomorphine may be controlled by domperidone or other antiemetic drugs. Often, patients on long-term apomorphine treatment are able to discontinue or decrease the dose of antiemetics without recurrence of these adverse effects.

The widespread application of apomorphine to control “on-off” fluctuations is limited by the necessity for subcutaneous administration. Alternative routes of administration have consequently been investigated. Intranasal apomorphine was shown to be effective in patients with Parkinson's disease but produced transient nasal blockage and burning sensation in two of five patients tested. Rectal administration of apomorphine has been shown to be effective and to have a longer duration of action than subcutaneously administered drug; however, higher doses of the drug are needed because of some first-pass metabolism. Furthermore, the delayed onset of action limits its application as a rescue therapy.

Sublingual administration of apomorphine has also been studied. Minimal first-pass metabolism allows for the use of lower doses compared with standard oral administration of apomorphine. In all studies, all patients (who were known to be responsive to subcutaneous apomorphine) fully “switched on”. The mean time to onset of effect was approximately 30 minutes and was comparable between the studies. The mean duration of action was longer following sublingual administration compared to subcutaneous administration. Unpleasant taste and inconsistency of dissolution were noted formulation problems.

The use of apomorphine in treating sexual dysfunction has also been investigated. For instance, the sublingual administration of apomorphine has been found in a clinical study to have a statistically significant effect on erectile dysfunction when compared with placebo (Dula et al Urology 2000; 56: 130-135). According to the literature, apomorphine promotes sexual function and performance because of the effect it exerts on the brain, in particular on the neurological mechanisms underlying sexual arousal. Apomorphine can thus be used to promote or enhance sexual function, treat sexual dysfunction, enhance libido and/or reduce impotence.

For optimal buccal absorption the apomorphine used should ideally be un-ionised at physiological pH. The pKa of apomorphine is 8.9 so, above a pH of about 9, significant amounts of the drug exist as free base. Conversely, at acidic pH (for example pH less than 4), the proportion of apomorphine as free base is negligible: almost all of the drug exists in an ionic charged form. Apomorphine in the charged state is poorly absorbed.

The proportion of drug which is un-ionised starts to increase significantly when the pH approaches 7; an alkaline pH yields increasing proportions of un-ionised drug. Thus, for optimal absorption, the drug should be in a non-acidic medium.

Apomorphine can undergo rapid oxidation if exposed to atmospheric oxygen or oxygen dissolved in a solvent such as water. Conventionally, this has been prevented by keeping aqueous solutions of the drug acidified. It is believed that commercially-available apomorphine for subcutaneous injection has a pH of about 3. Since this is intended for injection the pH does not influence systemic absorption. However, the nasal spray formulation described above is also an aqueous solution and is also believed to be acidic. This would imply that the formulation is not optimised for nasal absorption and the nasal irritation that has been reported might well derive from the acidic property of the formulation.

Administration of acidic apomorphine formulations into the mouth results in a stimulation of salivation. The excess saliva produced is rich in bicarbonate, which is intended to neutralise the acid and return the mouth to its normal, near neutral, pH. Although the resultant increase in pH should aid the absorption of apomorphine, there is also an increase in the amount of drug swallowed along with the additional volume of saliva. As a result, the amount of drug available for buccal absorption rapidly decreases.

Previous attempts have been made to make a formulation of apomorphine for buccal administration that is pharmaceutically stable.

WO 97/06786 discloses fast-dispersing dosage forms of apomorphine, including an acidified aqueous solution of apomorphine comprising gelatin and mannitol. A solid dosage form formed by freeze-drying the acidified aqueous solution, disintegrates when placed in the oral cavity.

WO 2006/120412 discloses a two-compartment system in which an aqueous solution of apomorphine is stabilized by the addition of acid and is held in one compartment and a suitable neutralizing buffer is held in a second compartment. Immediately before administration to the patient, the two liquids are mixed, the acidic apomorphine solution is neutralized and delivered to the mouth.

WO 2012/083269A1 discloses a gelatin film is made comprising acidified apomorphine in one layer and a neutralizing buffer in the other layer. When placed in the mouth, the gelatin dissolves, and the acidic apomorphine is neutralized rendering it suitable for absorption of the drug.

However, there remains a desire to optimise speed, efficacy and convenience of apomorphine-based treatments, particularly for the treatment of Parkinsons' disease, whilst overcoming issues concerning the stability of apomorphine.

### SUMMARY OF THE INVENTION

According to the invention, there is provided a composition comprising apomorphine or a pharmaceutically acceptable salt thereof.

The composition is preferably a liquid. The liquid may be a solution, dispersion or suspension.

The apomorphine or pharmaceutically acceptable salt thereof may be in the form of particles suspended in a liquid medium, such as a propellant. The particles may be solid particles. The particles may be insoluble in the liquid medium. However, in one embodiment, the composition is not in the form of a suspension, for example the composition may not comprise solid particles of apomorphine or pharmaceutically acceptable salt thereof.

Preferably, the composition is a solution. An advantage of providing a solution rather than a particle suspension is that the apomorphine or salt thereof is immediately available for absorption, for example via buccal or sublingual administration. Solid particles may have to dissolve in saliva before they can be absorbed, thus potentially delaying the time to peak concentration in a patient.

According to the invention, there is provided a composition comprising a solution of apomorphine or a pharmaceutically acceptable salt thereof.

If the composition comprises a solution of apomorphine, a solvent used to dissolve the apomorphine may be degassed to reduce or eliminate any oxygen that may be dissolved. This may be achieved by a number of conventional methods such as purging, using nitrogen gas. Consequently, a solution of the invention may comprise substantially no dissolved oxygen. A solution of the invention may be formed by using a solvent, such as a solvent comprising water, that has substantially no dissolved oxygen.

The composition may comprise water. The solution may be an aqueous solution. The composition may comprise, at least 5%, at least 10%, at least 20%, at least 30%, by weight, of water. The amount of water may be 5 to 50% by weight, such as 10 to 40% by weight.

According to the invention, there is provided a composition comprising apomorphine or a pharmaceutically acceptable salt thereof, and a solvent comprising water. Preferably, the solvent has been degassed to reduce or eliminate dissolved oxygen

The composition may comprise a non-aqueous solvent. The non-aqueous solvent may be as an alternative to, or in addition to, water.

The composition may comprise an excipient. The excipient may comprise a polymer. For example, the excipient may comprise polyethylene glycol (PEG), such as PEG400. The excipient may be present in an amount of at least 0.2% by weight, such as at least 0.5% by weight. For example, the excipient may be present in an amount of 0.2 to 2% by weight, such as 0.5 to 1% by weight. The excipient may assist with solubilisation and aerosol formation.

The composition may comprise substantially no water, or minimal amounts of water. For example, there may be less than 5%, less than 2%, less than 1%, less than 0.5%, less than 0.2%, or less than 0.1% by weight, of water.

The solution may be a non-aqueous solution.

According to the invention, there is provided a composition comprising apomorphine or a pharmaceutically acceptable salt thereof, and a non-aqueous solvent.

According to the invention, there is provided a composition comprising a non-aqueous solution of apomorphine, or a pharmaceutically acceptable salt thereof.

The non-aqueous solvent may comprise an organic solvent. Dissolving the apomorphine in the non-aqueous solvent may thus form the non-aqueous solution. The non-aqueous solvent preferably comprises a propellant. The composition may thus comprise the apomorphine dissolved in propellant. Preferably, the propellant comprises a hydrofluorocarbon (HFA). An example of a suitable propellant is HFA134a (1,1,1,2-Tetrafluoroethane). Other examples include HFA152a (1,1-Difluoroethane) and HFA227ea (1,1,1,2,3,3,3-Heptafluoropropane). A non-aqueous solution may thus be formed by dissolving the apomorphine or salt thereof, in the propellant.

The propellant may be present in the composition an amount of at least 20% by weight, at least 30%, at least 40% by weight, at least 50% by weight, at least 60% by weight, at least 70% by weight, at least 80% by weight, at least 90% by weight, or at least 95% by weight. The propellant may be present in an amount of up to 99% by weight.

According to the invention, there is provided a composition comprising apomorphine, or a pharmaceutically acceptable salt thereof, and a propellant.

According to the invention, there is provided a composition comprising a non-aqueous solution of apomorphine, or a pharmaceutically acceptable salt thereof, the composition comprising a propellant.

In one embodiment, the composition consists essentially of apomorphine or a pharmaceutically acceptable salt thereof, and a propellant.

In addition to the propellant, a non-aqueous co-solvent may be used to aid dissolution of the apomorphine or salt thereof. The non-aqueous co-solvent preferably comprises ethanol. The composition may thus comprise the apomorphine dissolved in propellant and co-solvent. Dissolving the apomorphine or salt thereof in the propellant and the co-solvent may thus form the non-aqueous solution. In one example, the composition may comprise HFA134a and ethanol. The amount of non-aqueous co-solvent (e.g. alcohol, such as ethanol), may be less than 50% by weight, or less than 35% by weight.

According to the invention, there is provided a composition comprising apomorphine or a pharmaceutically acceptable salt thereof, a propellant and a co-solvent.

According to the invention, there is provided a composition comprising a non-aqueous solution of apomorphine or a pharmaceutically acceptable salt thereof, the composition comprising a propellant and a co-solvent.

In one embodiment, the composition consists essentially of apomorphine or a pharmaceutically acceptable salt thereof, a propellant and a non-aqueous co-solvent.

In one embodiment, the composition may not comprise a co-solvent. For example, the composition may not comprise an alcohol. For example, the composition may not comprise ethanol.

In one embodiment, the composition may not comprise an anaesthetic. For example, the composition may not comprise lidocaine or prilocaine.

The applicant has appreciated the benefits of formulating apomorphine in such a way that it is optimised for administration to a mucous membrane, such as in in the oral cavity, e.g. by buccal or sublingual administration, allowing rapid absorption whilst being sufficiently stable to prevent auto-oxidation. This is particularly important because “on-off” phenomena in Parkinson's disease can occur very rapidly.

Surprisingly, it has been found that apomorphine, or a pharmaceutically acceptable salt thereof, can maintain stability, and resistance to oxidation, when dissolved in propellant with and without a co-solvent. This avoids the need to provide more complex delivery systems such as those described in WO 2006/120412 in which provisions were made to maintain the apomorphine in an acidic environment prior to administration. Consequently, compositions of the invention may not require the presence of an acid.

Compositions of the invention may have a pH of at least 4, preferably at least 6, (such as a pH of 6-8), more preferably at least 7. If the composition is a non-aqueous solution, when the composition is contacted with water (for example, when it is exposed to saliva in the oral cavity) the pH of the resulting solution may be at least 4, preferably at least 6 (such as a pH of 6-8), more preferably at least 7.

Formulation with a propellant means that it may be readily included in spray devices such as aerosol spray devices that are able to permit effective delivery by buccal administration.

The apomorphine may be present as the free base or as a pharmaceutically acceptable salt, such as an acid addition salt, e.g. hydrochloride salt.

Compositions of the invention may comprise at least 0.1% by weight apomorphine or pharmaceutically acceptable salt thereof. Compositions of the invention may comprise at least 0.5% by weight apomorphine or pharmaceutically acceptable salt thereof. For example, the amount of apomorphine in the composition may be 0.1% to 20% by weight, such as 0.5% to 15% by weight. Compositions of the invention may comprise at least 20% by weight of apomorphine or pharmaceutically acceptable salt thereof, at least 25% by weight of apomorphine or pharmaceutically acceptable salt thereof, or at least 30% by weight of apomorphine or pharmaceutically acceptable salt thereof. Compositions of the invention may comprise 1% to 50% by weight of apomporphine or pharmaceutically acceptable salt thereof.

Pharmaceutically acceptable derivatives of apomorphine are known. Examples include esters of apomorphine, such as diesters, e.g. diisobutyryl esters. Compositions of the invention, or for use in the invention, may include apomorphine derivatives (e.g. esters of apomorphine, or salts thereof) as an alternative, or in addition to, apomorphine (or salts thereof). However, derivatives of apomorphine (e.g. esters of apomorphine, or salts thereof) are less preferred. For example, diesters may be prodrugs which require enzymatic or chemical biotransformation to yield apomorphine in vivo. Administration of prodrugs may thus delay the pharmacological effect of apomorphine in the subject.

According to the invention, there is provided a method of forming a composition of the invention, comprising combining or mixing apomorphine, or a pharmaceutically acceptable salt thereof, with a non-aqueous solvent. The method may comprise forming a non-aqueous solution. The method may comprise combining the apomorphine, or pharmaceutically acceptable salt thereof, with a propellant. The method may be carried out substantially in the absence of air or oxygen. The method may comprise adding the non-aqueous solution to a container, preferably substantially in the absence of air or oxygen.

According to the invention, there is provided a method of forming a composition of the invention, comprising combining or mixing apomorphine, or a pharmaceutically acceptable salt thereof, with a solvent comprising water. The solvent has preferably been degassed to reduce or eliminate dissolved oxygen. The method may comprise forming an aqueous solution. The method may comprise combining the apomorphine, or pharmaceutically acceptable salt thereof, with a propellant. The method may be carried out substantially in the absence of air or oxygen. The method may comprise adding the aqueous solution to a container, preferably substantially in the absence of air or oxygen. The method may comprise degassing the solvent to reduce or eliminate the dissolved oxygen.

Preferably, the propellant is added to the container following substantially no, or minimal, exposure of the propellant to air.

Compositions of the invention may be substantially free of oxygen.

Compositions of the invention may comprise a chelating agent and/or antioxidant, such as sodium metabisulfite. Alternatively, compositions of the invention may be substantially free of chelating agent and/or antioxidant, such as sodium metabisulfite.

According to the invention, there is provided a container or canister comprising a composition of the invention.

The container or canister is preferably airtight and thus substantially impermeable to air and/or oxygen ingress. The container may be substantially impermeable to water, e.g. moisture. Preferably, the container comprising the composition is substantially free of air, or oxygen. The container may be substantially free of water, e.g. moisture. The canister may contain an inert gas, such as nitrogen.

The container or canister may be a canister manufactured from a metal such as aluminium. Alternatively, the container or canister may be manufactured from a plastics material such as PET (Polyethylene terephthalate).

The container or canister may be a canister manufactured from a suitable material and coated with a substance known to prevent air or oxygen ingress. An example of such a coating material is polytetrafluoroethylene or PTFE.

The container or canister may comprise an outlet for dispensing the composition. For instance, the container may comprise a valve. The valve may enable dispensing of a pre-determined volume of the composition from the container or canister. For example, the valve may be a metering valve.

The container or canister may be compatible with a dispenser or actuator, for dispensing the composition from the container or canister. For example, the container or canister may be releasably engagable with the dispenser or actuator. The dispenser or actuator may provide the means for opening the valve in the container or canister, such as an actuator nozzle.

The container or canister, and dispenser or actuator, may combine to form a dispensing device, such as a dispensing device of the invention.

According to the invention, there is provided a kit comprising a composition of the invention. The kit may comprise a) a container or canister of the invention; and b) a dispenser or actuator for dispensing a composition from the container. The container or canister is preferably releasably engagable with the dispenser or actuator. The container/canister and dispenser/actuator may be separate. Once the contents of the container/canister has depleted, the container/canister may be replaced.

According to the invention, there is provided a dispensing device, the device comprising a composition of the invention. Preferably, the device is a spray device e.g. an aerosol spray device. The device may be configured to dispense a predetermined dose of the composition.

The device may be a pressurised metered dose device. For example, the device may comprise a container or canister comprising the composition, and a dispenser or actuator for dispensing the composition from the container or canister. The container or canister may releasably engage with the dispenser or actuator. In one embodiment, the container or canister comprises a metering valve and the actuator comprises an actuator nozzle. In use, the valve engages the actuator and when the container or canister is depressed relative to the actuator, the actuator nozzle urges the valve into on open configuration to permit the composition to be dispensed, preferably as an aerosol.

In one embodiment, the kit or device may be configured to dispense a dose of 0.05 mg to 100 mg of apomorphine or pharmaceutically acceptable salt thereof, dose. For example, one application, or one spray, may dispense 0.05 mg to 100 mg of apomorphine or pharmaceutically acceptable salt thereof. Alternatively, the kit or device may be configured to dispense a dose of 0.05 mg to 75 mg, 0.1 mg to 50 mg or 1 mg to 40 mg apomorphine or pharmaceutically acceptable salt thereof.

Preferably, the kit or device is configured to deliver particles or droplets with particle size that are not in the respirable range, thereby permitting a buccal or sublingual spray to be delivered without the risk of inhalation.

The kit or device may be configured to deliver particles or droplets of the composition, having a mean diameter greater than 10 μm. For example, the mean diameter may be 20 μm or greater. The mean diameter may be 50 μm or greater.

The kit or device may be configured to deliver particles or droplets of the composition, having less than 10% of particles less than 10 μm in diameter. There may be less than 5% of particles less than 10 μm in diameter.

The kit or device may be configured to deliver particles or droplets of the composition, having a mass median aerodynamic diameter (MMAD) greater than 10 μm. MMAD refers to the diameter at which 50% of the particles or droplets by mass are larger and 50% are smaller. In order for particles or droplets to be carried into the lungs, they must be extremely fine, for example having a MMAD of less than 10 μm. The MMAD may be at least 20 μm. The MMAD may be at least 50 μm.

The kit or device may be configured to deliver particles or droplets comprising a volume median diameter (VMD) greater than 10 μm. The Volume Median Diameter (VMD) refers to the midpoint droplet size (median), where half of the volume of spray is in droplets smaller, and half of the volume is in droplets larger than the median. The VMD may be at least 20 μm. The VMD may be at least 50 μm.

The kit or device may be configured to deliver particles or droplets of the composition having a fine respirable fraction or fine particle fraction (FPF) of less than 30%, preferably less than, 20%, more preferably less than 10%. The FPF refers to the proportion of emitted particles or droplets that have a diameter smaller than 5 μm, the respirable dose.

A number of methods are available to determine the size distribution of particles or droplets. Cascade impactors such as the Anderson Cascade Impactor or Next Generation Impactor (NGI), can be used to obtain the size distribution of an aerosol. NGI is a cascade impactor for classifying aerosol particles into size fractions, containing seven impaction stages plus a final micro-orifice collector, which is commercially available, for example, from MSP Corporation, MN, USA. An example of such an impactor is described, for example in U.S. Pat. No. 6,595,368.

Particle/droplet size can be measured by laser diffraction techniques. For instance, light from a laser may be directed into a cloud of particles/droplets, which are suspended in a transparent gas such as air. The particles/droplets scatter the light; smaller particles/droplets scattering the light at larger angles than bigger particles/droplets. The scattered light can be measured by a series of photodetectors placed at various angles. This is known as the diffraction pattern for the sample. The diffraction pattern can be used to measure the size of the particles/droplets. Particle diameters may be calculated from the measured volume of the particles/droplets, assuming a sphere of equivalent volume.

According to the invention, there is provided a dispensing device comprising a composition of the invention (e.g., a composition comprising apomorphine or a pharmaceutically acceptable salt thereof) which is configured to deliver the composition in form of particles or droplets, such as an aerosol. The particles or droplets may have:

i) a mean diameter greater than 10 μm (e.g. 20 μm or greater, or 50 μm or greater);

ii) less than 10% of particles less than 10 μm in diameter (e.g. less than 5% of particles less than 10 μm in diameter.

iii) a MMAD greater than 10 μm (e.g. 20 μm or greater, or 50 μm or greater);

iv) a volume median diameter (VMD) greater than 10 μm (e.g. 20 μm or greater or 50 μm or greater); and/or
v) a fine particle fraction (FPF) less than 30%, preferably less than, 20%, more preferably less than 10%.

An example of a device that may be suitable for administration of a composition of the invention is described in US2018/0344950 A1. Other examples include conventional nasal spray bottles, which permit atomisation of solutions contained therein, by manual application of pressure, such as by squeezing the bottle or by using a pump.

According to the invention, there is provided a composition of the invention (e.g. a composition comprising apomorphine or a pharmaceutically acceptable salt thereof), for use as a medicament.

According to the invention, there is provided a composition according to the invention, for use in treating Parkinson's disease in a subject.

According to the invention, there is provided use of a composition according to the invention, in the manufacture of a medicament for use in treating Parkinson's disease in a subject.

According to the invention, there is provided a method of treating Parkinson's disease comprising administering a composition of the invention to a subject in need of such treatment. The subject is preferably a human.

Alternatively, compositions of the invention may be used to promote or enhance sexual function, treat sexual dysfunction, enhance libido and/or reduce impotence. For example, compositions of the invention may be used to treat Male Erectile Dysfunction.

The composition may be administered for pre-gastric absorption of the active ingredient, that is, absorption of the active ingredient from that part of the alimentary canal prior to the stomach. The term “pre-gastric absorption” thus includes buccal, sublingual, oropharyngeal and oesophageal absorption. Administration may be topical, by administration to a mucous membrane. In a preferred example, the composition may be administered to the oral cavity, for example by buccal administration. Alternatively, the composition may be administered to the nasal cavity.

The apomorphine, or salt thereof, is preferably formulated in such a way that it is optimised for oral administration e.g. buccal administration, and hence rapid absorption. This is particularly important because “on-off” phenomena in Parkinson's disease can occur very rapidly.

The composition may be administered to subject to provide 0.05 mg to 100 mg of apomorphine. Alternatively, the composition may be administered to the subject to provide 0.05 mg to 75 mg, 0.1 mg to 50 mg or 1 mg to 40 mg apomorphine or pharmaceutically acceptable salt thereof.

The frequency of dose may be dependent on the frequency of the subject's condition. For example, it may depend on the frequency of a patient's “on-off” fluctuations who is suffering from Parkinson's disease. An aforementioned dose may be administered each time a patient has an “off” period, for example at the onset of each off period.

In the context of treating sexual dysfunction (such as Male Erectile Dysfunction), the frequency of dose may depend on the desired sexual activity of the subject. For example, an aforementioned dose may be administered prior to engaging in sexual activity. For example, it may be administered within one hour, 30 minutes, 15 minutes, 10 minutes or 5 minutes prior to engaging in sexual activity.

The composition may be administered to the subject as particles or droplets, such as an aerosol. The particles or droplets may have:

i) a mean diameter greater than 10 μm (e.g. 20 μm or greater, or 50 μm or greater); and/or ii) less than 10% of particles less than 10 μm in diameter (e.g. less than 5% of particles less than 10 μm in diameter; and/or

iii) a MMAD greater than 10 μm (e.g. 20 μm or greater, or 50 μm or greater); and/or iv) a volume median diameter (VMD) greater than 10 μm (e.g. 20 μm or greater or 50 μm or greater); and/or

v) a fine particle fraction (FPF) less than 30%, preferably less than, 20%, more preferably less than 10%.

According to the invention, there is provided method comprising atomising a composition of the invention, (e.g. a composition comprising apomorphine, or a pharmaceutically acceptable salt thereof). The method may comprise forming an aerosol. Particles or droplets formed may have:

i) a mean diameter greater than 10 μm (e.g. 20 μm or greater, or 50 μm or greater); and/or ii) less than 10% of particles less than 10 μm in diameter (e.g. less than 5% of particles less than 10 μm in diameter; and/or

iii) a MMAD greater than 10 μm (e.g. 20 μm or greater, or 50 μm or greater); and/or iv) a volume median diameter (VMD) greater than 10 μm (e.g. 20 μm or greater or 50 μm or greater); and/or

v) a fine particle fraction (FPF) less than 30%, preferably less than, 20%, more preferably less than 10%.

### EXAMPLES

Preparation of Formulations

### Example 1

Formulations of apomorphine were prepared at a 1.0 and 10% concentration in HFA 134a propellant. The details of these preparations are shown in Table 1.

TABLE 1
Details of manufactured formulation systems.

| | Apomorphine | Ethanol | Acid | HFA 134a |
|---|---|---|---|---|
| Formulation | Concentration | Concentration | Concentration | Concentration |
| Batch | (% w/w) | (% w/w) | (% w/w) | (% w/w) |
| NP-AP-94-C070 | 1.0 | 0.0 | 0.0 | 99.0 |
| NP-AP-94-C075 | 10.0 | 0.0 | 0.0 | 90.0 |

In the case of manufacture of drug-only formulations with HFA134a, the required amount of drug was weighed into the MDI canister and a 50 μL with Butyl elastomer (Bespak, Kings Lynn, UK) was crimped on to the canister. The HFA was filled through the valve and formulation was sonicated for 20-mins. The canisters were then placed valve down for 14-days to laager.

The chemical stability of the different apomorphine formulations manufactured in HFA 134a at T-zero, 24 and 72-hours is shown in Table 2.

TABLE 2
Chemical stability of the different HFA-based
formulations at T-zero, 24 and 72-hours.

| | T-zero | | 24-hours | | 72-hours | |
|---|---|---|---|---|---|---|
| | | Total | | Total | | Total |
| | Assay | Imps. | Assay | Imps. | Assay | Imps. |
| Canister | (%) | (%) | (%) | (%) | (%) | (%) |
| NP-AP-94-C070 | 99.89 | ND | 99.67 | ND | 99.22 | ND |
| NP-AP-94-C075 | 97.45 | 0.08 | 90.45 | 0.29 | 89.98 | 0.55 |

Formulations prepared at 1.0% w/w concentration in HFA134a were extremely stable. There was no indication of impurities being generated up to 72-hours. In the case of the 1.0% w/w formulations they had passed the acceptance criteria as no individual impurity has greater than 0.2% and the sum of all unknown impurities was below 2.0%.

For the 10% API and HFA134a formulation there was increase in the measured impurities overtime as at 72-hours had increased to 0.55% w/w. There was no individual impurity greater than 0.2% and since the total impurities was less than 2.0% the formulation was within specification.

Hence, these data suggest that all formulations were chemically stable in HFA 134a.

### Example 2

Formulations of apomorphine were prepared at a 30% concentration in HFA 134a propellant with and without an excipient, polyethylene glycol 400 (PEG400). The details of these preparations are shown in Table 3. The aqueous solvent was degassed prior to its use, to eliminate dissolved oxygen and the formulations were made in the absence of oxygen.

TABLE 3
Details of manufactured formulation systems.

| | Apomorphine | Excipient | Aqueous | HFA 134a |
|---|---|---|---|---|
| Formulation | Concentration | (PEG400; | Component | Concentration |
| Batch | (% w/w)* | % w/w) | (% w/w) | (% w/w) |
| NP-94-149-001 | 30 | — | 35 | 35 |
| NP-94-149-004 | 30 | 0.7 | 20.65 | 48.65 |

*A 30% concentration equates to a delivered dose of 23.2 mg of apomorphine via a 75 μl valve.

The composition was added to an MDI canister.

The chemical stability of the different apomorphine formulations manufactured in HFA 134a at T-zero, 28-, 32-, 36-, 40-, and 48-days is shown in Table 4.

TABLE 4
Chemical stability of the different HFA-
based formulations for up to 48 days.

| | 0-days | | 28-days | | 32-days | |
|---|---|---|---|---|---|---|
| | | Total | | Total | | Total |
| | Assay | Imps. | Assay | Imps. | Assay | Imps. |
| | (%) | (%) | (%) | (%) | (%) | (%) |
| NP-94-149-001 | 99.75 | ND | 99.45 | ND | 99.14 | ND |
| NP-94-149-004 | 100.12 | ND | 99.19 | ND | 99.83 | ND |
| | 36-days | | 40-days | | 48-days | |
| | | Total | | Total | | Total |
| | Assay | Imps. | Assay | Imps. | Assay | Imps. |
| | (%) | (%) | (%) | (%) | (%) | (%) |
| NP-94-149-001 | 99.14 | ND | 99.28 | ND | 98.99 | ND |
| NP-94-149-004 | 99.49 | ND | 98.97 | ND | 98.12 | 0.08 |

The formulations prepared at 30% concentration in HFA 134a propellant were extremely stable for up to 48 days despite not being acidified.

1. A composition comprising a solution of apomorphine, or a pharmaceutically acceptable salt thereof, the composition comprising a propellant, wherein the solution: i) is a non-aqueous solution; or ii) comprises degassed water.

2. A composition according to claim 1, wherein the propellant comprises a hydrofluorocarbon (HFA).

3. A composition according to claim 2, wherein the propellant comprises HFA-134a.

4. A composition according to any preceding claim comprising a co-solvent.

5. A composition according to claim 4, wherein the co-solvent comprises an organic solvent.

6. A composition according to claim 5, wherein the organic solvent comprises an alcohol, preferably ethanol.

7. A composition according to any preceding claim, wherein the composition comprises an excipient.

8. A composition according to claim 7, wherein the excipient is a polymer.

9. A composition according to claim 8, wherein the excipient is polyethylene glycol.

10. A composition according to any of claims 7 to 9, wherein the excipient is present in an amount of 0.2% to 2% by weight.

11. A composition according to any preceding claim, in which the apomorphine is present in an amount of 1 to 50% by weight.

12. A composition according to claim 11, in which the apomorphine is present in an amount of at least 5%, 10%, 20% or 30% by weight.

13. A composition according to any preceding claim in which the solution of apomorphine comprises water, and the amount of water in the composition is 10 to 40% by weight.

14. A composition according to any preceding claim, in which the propellant is present in an amount of at least 30% by weight, optionally up to 99% by weight of the composition.

15. A kit comprising: a) a canister comprising a composition as defined in any preceding claim; and b) an actuator for dispensing the composition from the canister.

16. A kit according to claim 15 which is configured to deliver the composition in the form of particles or droplets having a mass median aerodynamic diameter (MMAD) or volume median diameter (VMD) greater than 10 μm; and/or a fine particle fraction (FPF) less than 30%.

17. A kit comprising a) a canister; and b) an actuator for dispensing the composition from the canister, wherein the canister comprises a composition, the composition comprising apomorphine or a pharmaceutically acceptable salt thereof, wherein the kit is configured to deliver the composition in the form of particles or droplets having a mass median aerodynamic diameter (MMAD) or volume median diameter (VMD) greater than 10 μm; and/or a fine particle fraction (FPF) less than 30%

18. A kit according to claim 17, wherein i) the apomorphine or pharmaceutically acceptable salt thereof is in solution, optionally i) a non-aqueous solution; or ii) a solution comprising degassed water; and/or b) the composition comprises a propellant.

19. A kit according to according any of claims 15 to 18, wherein the canister comprises a metering valve.

20. A kit according to any of claims according to any of claims 15 to 19, wherein the kit is configured to deliver a pre-determined, preferably wherein the dose is 0.05 mg to 100 mg of apomorphine or pharmaceutically acceptable salt thereof.

21. A dispensing device comprising a composition as defined in any of claims 1 to 14.

22. A device according to claim 21, which is configured to deliver the composition in the form of particles or droplets having a mass median aerodynamic diameter (MMAD) or volume median diameter (VMD) greater than 10 μm; and/or a fine particle fraction (FPF) less than 30%.

23. A dispensing device comprising a composition, the composition comprising apomorphine or a pharmaceutically acceptable salt thereof, wherein the device is configured to deliver the composition in form of particles or droplets having a mass median aerodynamic diameter (MMAD) or volume median diameter (VMD) greater than 10 μm; and/or a fine particle fraction (FPF) less than 30%.

24. A device according to claim 23, wherein i) the apomorphine or pharmaceutically acceptable salt thereof is in solution, optionally i) a non-aqueous solution or ii) a solution comprising degassed water; and/or b) the composition comprises a propellant,

25. A device according any of claims 21 to 24 which is a spray device, preferably a pressurised metered dose dispensing device for dispensing a predetermined dose of the composition.

26. A device according to claim 25, which comprises: a) a container comprising the composition, preferably wherein the container comprises a metering valve; and b) an actuator for dispensing the composition from the container.

27. A device according to any of claims 21 to 26, which is configured to deliver a predetermined dose, preferably wherein the predetermined dose is 0.05 mg to 100 mg of apomorphine or pharmaceutically acceptable salt thereof.

28. A composition according to any of claims 1 to 14, for use as a medicament.

29. A composition according to any of claims 1 to 14, for use in treating Parkinson's disease or Male Erectile Dysfunction.

30. The composition for the use according to claim 29, wherein the composition is administered to the subject topically, preferably by buccal administration.

31. The composition for the use according to claim 29 or claim 30 which is administered to the subject in the form of particles or droplets having i) a mass median aerodynamic diameter (MMAD) or volume median diameter (VMD) greater than 10 μm; and/or a fine particle fraction (FPF) less than 30%.

32. A composition for use in treating Parkinson's disease in a subject, the composition comprising apomorphine, or a pharmaceutically acceptable salt thereof, wherein the composition is administered in the form of particles or droplets having a mass median aerodynamic diameter (MMAD) or volume median diameter (VMD) greater than 10 μm; and/or a fine particle fraction (FPF) less than 30%.

33. A composition for the use according to claim 32, wherein the composition is administered to the subject topically, preferably by buccal administration,

34. The composition for the use according to claim 32 or claim 33, wherein the apomorphine or pharmaceutically acceptable salt thereof is in solution, optionally i) a non-aqueous solution, or ii) a solution comprising degassed water; and/or wherein the composition comprises a propellant.

35. A method comprising atomising a composition, the composition comprising apomorphine, or a pharmaceutically acceptable salt thereof, to form particles or droplets having a mass median aerodynamic diameter (MMAD) or volume median diameter (VMD) greater than 10 μm; and/or a fine particle fraction (FPF) less than 30%.

36. A method according to claim 35, wherein the apomorphine or pharmaceutically acceptable salt thereof is in solution, optionally i) a non-aqueous solution, or ii) a solution comprising degassed water; and/or wherein the composition comprises a propellant.
